(19) United States
(12) Patent Application Publication
(10) Pub. No.: US 2026/0294505 A1
(43) Pub. Date: Oct. 1, 2026

(54) HANDHELD BATTERY-POWERED VAPOR DELIVERY DEVICES AND METHODS

(71) Applicant: John H. SHADDUCK, Menlo Park, CA (US)

(72) Inventor: John H. SHADDUCK, Menlo Park, CA (US)

(21) Appl. No.: 19/532,017

(22) Filed: Feb. 6, 2026

Related U.S. Application Data
(60) Provisional application No. 63/779,746, filed on Mar. 28, 2025.

(51) Int. Cl. A61B 18/04; A61B 18/00; A61B 18/08

(52) U.S. Cl. CPC A61B 18/04; A61B 18/082; A61B 2018/00327; A61B 2018/00577; A61B 2018/00625; A61B 2018/048

(57) ABSTRACT

A handheld medical device, system, and method are disclosed for generating and delivering a condensable vapor medium to targeted tissue for localized thermal treatment. The device comprises an ergonomic handle, an elongate introducer having a working end configured for positioning within a body cavity or adjacent tissue, and a vapor-generating subsystem including a resistive heating structure thermally coupled to a liquid flow path to convert a metered flow of liquid medium into condensable vapor. A liquid delivery subsystem supplies the liquid at controlled low flow rates, and an onboard battery serves as an exclusive power source to enable fully portable operation without external generators or cords. A controller coordinates liquid delivery and heating to deliver vapor in controlled pulses or delivery intervals while reducing liquid droplet carryover, optionally using flow restrictors and/or vapor conditioning structures to suppress two-phase flow instabilities. The working end may include one or more vapor outlets, deployable vapor-delivery needles, or surface-contact applicators for penetrating or non-penetrating delivery. In an exemplary otolaryngology application, the device may be positioned within the nasal cavity to deliver condensable vapor adjacent posterior nasal nerve branches for treatment of rhinitis and related upper-airway conditions

### CROSS-REFERENCE TO RELATED APPLICATION

This is a non-provisional of U.S. Provisional Application No. 63/779,746 filed Mar. 28, 2025, the entirety of which is incorporated by reference.

### FIELD OF THE DISCLOSURE

The disclosure relates generally to vapor-based tissue treatment technologies and, more particularly, to handheld, self-contained medical devices, systems, and methods for generating and delivering condensable vapor to biological tissue for controlled thermal modification of targeted tissue.

### BACKGROUND OF THE DISCLOSURE

Rhinitis generally refers to inflammation or dysfunction of tissues lining the nasal cavity and is commonly associated with symptoms including excessive mucous secretion, nasal congestion, post-nasal drip, sneezing, itching, and nasal irritation. Chronic rhinitis, including allergic, non-allergic, and vasomotor forms, affects a substantial patient population and may persist despite prolonged medical therapy. In many patients, chronic rhinitis is associated with abnormal neural signaling to the nasal mucosa, including parasympathetic input that contributes to glandular hypersecretion, vascular engorgement, and mucosal edema.

Conventional management of chronic rhinitis typically involves pharmacologic therapies such as antihistamines, topical or systemic corticosteroids, adrenergic agents, and anticholinergic medications. These therapies often require long-term or indefinite use and may be ineffective, poorly tolerated, or insufficient to control symptoms in certain patient populations. As a result, there remains a need for procedural approaches that modulate underlying neural, glandular, or submucosal contributors to rhinitis in a controlled and durable manner.

Anatomically, the nasal cavity contains multiple neural, vascular, glandular, and lymphoid structures that influence nasal airflow, secretion, and sensation. In particular, branches of posterior nasal nerves extend to the turbinates and adjacent mucosal tissues, where parasympathetic signaling may contribute to symptoms of chronic rhinitis. Targeted modification of such neural structures, as well as selective treatment of adjacent submucosal, glandular, or hypertrophic tissues, may provide therapeutic benefit while preserving surrounding anatomy.

### SUMMARY OF THE DISCLOSURE

The present invention relates generally to handheld medical devices, systems, and methods for generating and delivering a condensable vapor medium to targeted tissue. In particular embodiments, the disclosed technology provides a self-contained, portable vapor-generation and delivery platform configured to deliver controlled thermal energy through phase-change of a vapor medium. The devices and methods described herein may be used to achieve localized tissue modification, ablation, desensitization, coagulation, remodeling, or volume reduction in a wide range of clinical applications.

In exemplary embodiments, the disclosed system generates a high-quality condensable vapor from a controlled flow of liquid medium and delivers the vapor to a target tissue site through a minimally invasive delivery interface. Upon contacting tissue, the vapor condenses and releases latent heat, thereby depositing thermal energy efficiently and locally. The use of condensable vapor enables rapid energy transfer while allowing treatment parameters such as vapor dose, pulse duration, and delivery location to be precisely controlled.

In one aspect, the invention provides a handheld vapor-delivery device comprising: an ergonomic handle configured for single-hand operation; an elongate introducer extending from the handle to a working end configured for positioning within a body cavity or adjacent tissue; a vapor-generating subsystem including a resistive heating structure configured to convert a metered flow of liquid medium into a condensable vapor; a liquid delivery subsystem configured to supply the liquid medium to the heating structure at low and controlled flow rates; and an onboard battery serving as the exclusive power source, thereby enabling fully portable operation without reliance on external power generators, cords, or consoles.

In certain embodiments, the resistive heating structure comprises an electrically energized resistive element thermally coupled to tubing with a liquid flow path therein, such that a liquid medium flowing through the heating region is rapidly vaporized. The heating structure may be located within the handle, within an introducer near the working end, or in a hybrid arrangement including multiple heating stages. Such configurations may improve vapor quality, reduce thermal losses, minimize dead volume, and enhance energy efficiency under battery-powered operation.

The working end of the device may include one or more vapor outlets configured to emit vapor directly to tissue surfaces and/or through one or more deployable vapor-delivery needles. The delivery interface may be configured for penetrating delivery, non-penetrating surface delivery, or combinations thereof. In some embodiments, the working end includes a tissue-contact applicator having a shaped perimeter and multiple vapor ports to distribute vapor over a selected tissue area.

The device may further include a controller configured to coordinate operation of the liquid delivery subsystem and the resistive heating structure to deliver vapor in controlled pulses or delivery intervals. The controller may regulate liquid flow, heating power, and timing to achieve repeatable vapor generation while reducing liquid droplet carryover. In certain embodiments, flow stabilization features such as proximal flow restrictors, expansion regions, pressure regulation elements, or vapor conditioning structures may be provided to suppress two-phase flow instabilities and promote uniform delivery of high-quality vapor.

In one exemplary otolaryngology (ENT) embodiment, the handheld vapor-delivery device is introduced into a patient's nasal cavity and positioned at or near a target tissue region adjacent a turbinate, lateral nasal wall, or other anatomical structure. In such applications, the device may be used to deliver condensable vapor to tissue regions proximate to the branches of the posterior nasal nerves, including posterior superior lateral nasal nerves and posterior inferior lateral nasal nerves, which extend toward the superior, middle, and inferior turbinates and associated mucosal tissues. These tissues contain glands, vasculature, and neural elements involved in secretion, humidification, and airflow regulation. By selectively delivering thermal energy via condensation of a vapor medium near such neural structures, parasympathetic signaling to the nasal mucosa may be reduced or interrupted, thereby decreasing mucous hypersecretion, congestion, and related symptoms.

Although posterior nasal nerve modulation for treatment of chronic rhinitis represents a principal example, the invention is not limited to that indication. The disclosed vapor-based devices and methods may be used to selectively treat a variety of nasal, nasopharyngeal, or upper-airway tissues where controlled thermal delivery is desirable. By way of non-limiting example, the technology described herein may be applied to turbinate hypertrophy reduction, submucosal tissue remodeling to improve airflow, septal swell body reduction, nasal valve obstruction treatment, vasomotor or non-allergic rhinitis, allergic rhinitis using nerve-targeting or submucosal approaches, adenoid or tonsillar hypertrophy reduction, nasopharyngeal lymphoid tissue modification, chronic post-nasal drip associated with neurogenic or glandular targets, eustachian tube dysfunction involving tissue adjacent the tubal opening, soft palate or supraglottic tissue modification, and other upper-airway tissue treatments.

In all such embodiments, the disclosed vapor-based platform enables precise control of vapor generation, delivery timing, and energy deposition in a compact, handheld, battery-powered format. Variations in device geometry, vapor generation architecture, delivery interface configuration, and clinical treatment application are within the scope of the invention.

These and other aspects, embodiments, and advantages of the invention will become apparent from the following detailed description and accompanying drawings.

### BRIEF DESCRIPTION OF THE FIGURES

FIG. 1 is a transparent view of a battery-based handheld vapor treatment device configured for ablation of posterior nasal nerve function for the treatment of rhinitis.

FIG. 2 is an enlarged view of the working end of the device of FIG. 1 illustrating a vapor delivery needle for penetrating tissue.

FIG. 3A is an enlarged view of the resistive heating assembly of FIG. 1.

FIG. 3B is an exploded view of the resistive heating assembly of FIG. 3A.

FIG. 4A is a transparent view of another battery-based handheld vapor treatment device configured for ablation of posterior nasal nerve function with a distal resistive heating assembly, an insertable pump assembly, and an insertable syringe cartridge carrying a liquid medium removed from the device.

FIG. 4B is another transparent view of the device of FIG. 4A with the insertable pump assembly and the insertable syringe cartridge carrying the liquid medium inserted into the device.

FIG. 4C is an enlarged view of the resistive heating assembly of FIGS. 4A and 4B.

FIG. 5A is an enlarged, partly sectional view of the insertable pump assembly and the insertable syringe cartridge carrying the liquid medium.

FIG. 5B is a sectional view of an insertable pump assembly and a collapsible bellows carrying a liquid medium.

FIG. 6 is a schematic view of another handheld vapor treatment device configured with a primary resistive heating assembly in the handle and a secondary resistive heating assembly in the distal region of an introducer member.

FIG. 7 is a view of a resistive heating assembly similar to that of FIG. 4C with a proximal flow restrictor and a distal enlarged expansion zone adapted for stabilizing vapor generation and vapor quality.

FIG. 8A is a view of a working end of a device similar to that of FIG. 2 with a different needle variation.

FIG. 8B is a view of another working end similar to that of FIG. 8A with a plurality of vapor delivery needles.

FIG. 8C is a view of another working end similar to that of FIGS. 8A-8B with a variation of multiple vapor delivery needles

FIG. 9 is a sectional view of a human nasal cavity showing the turbinates and posterior nasal nerves targeted for ablation to impair or desensitize their function with the device of FIG. 4B introduced into an intraluminal position to ablate a PN nerve.

FIG. 10 is an enlarged perspective view of a device working end with a needle carrying a capacitance sensing electrode arrangement.

FIG. 11 is an enlarged view of a device working end with an extendable-retractable needle.

FIG. 12A is an enlarged view of a device working end with a concave surface and a vapor outlet.

FIG. 12B is a view of a device working end with an elongated concave surface with a plurality of vapor outlets.

FIG. 13 is a transparent view of another variation of a handheld vapor device similar to that of FIG. 1, including inflow and outflow channels for circulating vapor medium through the working end and a valve for directing vapor medium flow to the needle.

FIG. 14A is an enlarged schematic view of the inflow and outflow channels of FIG. 13, showing vapor medium circulating through the channels with the valve in a first position.

FIG. 14B is a view of the inflow and outflow channels of FIG. 14A showing the vapor medium being emitted from the needle with the valve in a second position.

FIG. 15 is a schematic view of a wire-type resistive heater positioned in a flow channel.

### DETAILED DESCRIPTION OF THE DISCLOSURE

This section describes exemplary embodiments of handheld vapor-based ablation devices configured for use in ENT procedures. The handheld device is configured to generate and deliver condensable vapor to tissue without reliance on an external power source. The device may include a handle sized and shaped for one-handed operation. The features described herein may be implemented individually or in combination in various device configurations and are not intended to be limiting. Although many embodiments described herein employ a vapor-delivery needle, other embodiments may deliver vapor through a fixed working end, a deployable outlet structure, a diffuser, a surface treatment structure, or other vapor-delivery interfaces. The invention is not limited to needle-based vapor delivery or surface treatment devices unless expressly recited in the claims.

### Exemplary Handheld Vapor-Based Treatment Devices

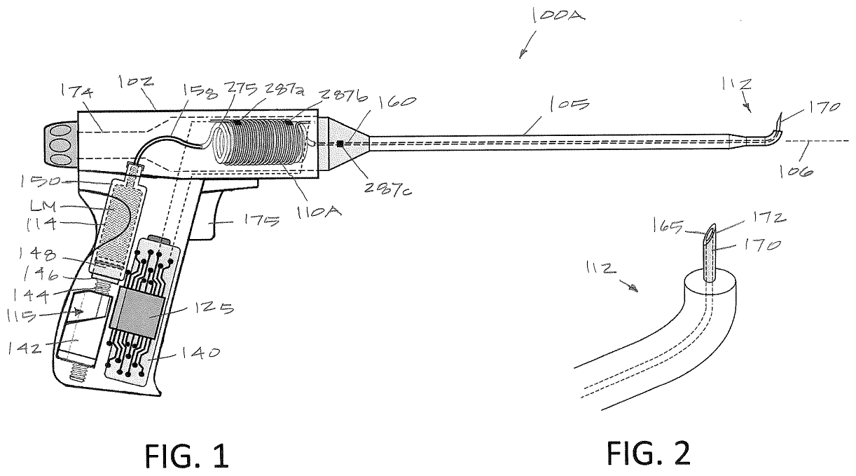

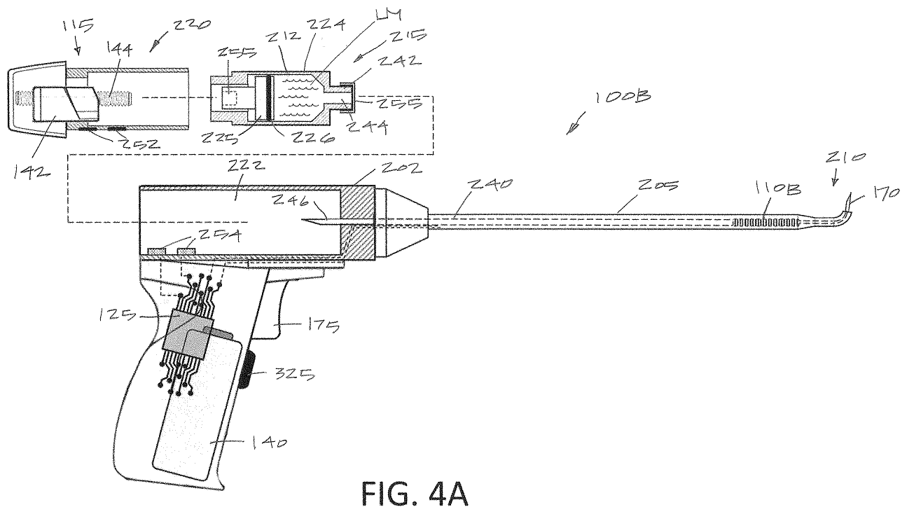

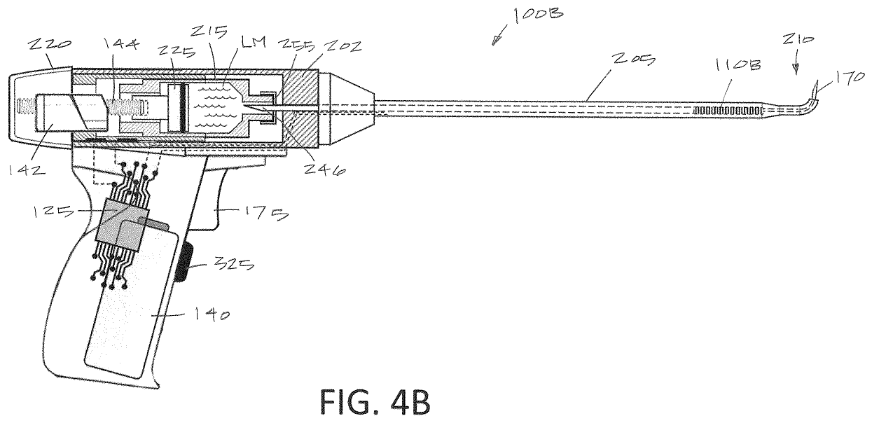

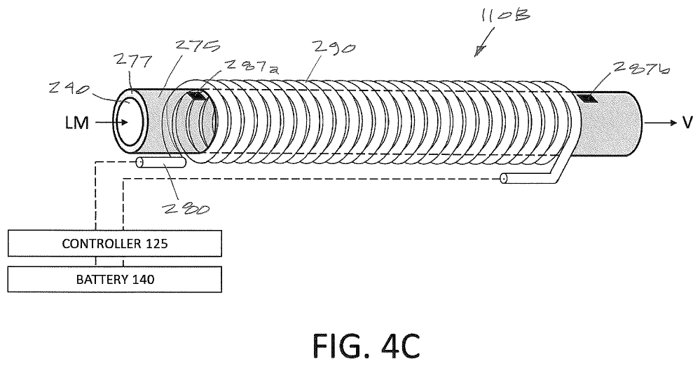

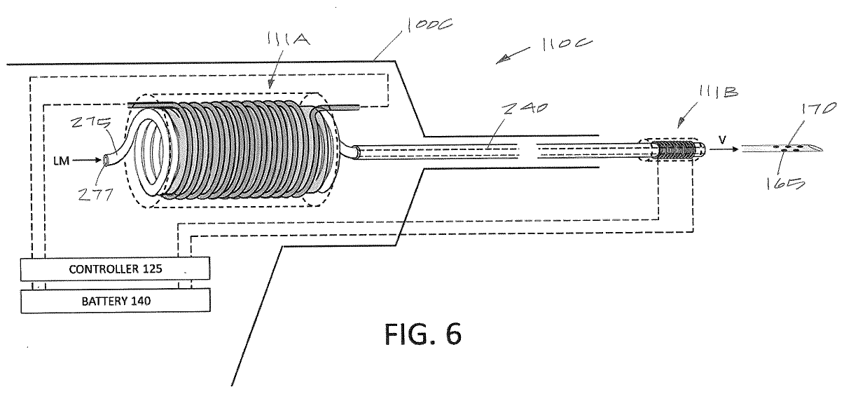

FIGS. 1-3B illustrate a first embodiment of a handheld vapor-delivery device 100A with handle 102 coupled to an elongate introducer member 105 extending about axis 106 with a vapor-generating subsystem including a resistive heating structure 110A fixed in place in the handle. FIGS. 4A-4C illustrate a second embodiment of handheld vapor-delivery device 100B where components of the vapor-generating subsystem are removable and insertable with a resistive heating structure 110B carried proximate a distal portion of the introducer member 105 instead of in the handle. FIG. 6 schematically illustrates a further variation of a vapor delivery device 110C with the resistive heating assembly 110C comprising primary and secondary resistive heating structures 111A, 111B. In all aspects, the functional subsystems, components and operating parameters of the various embodiments are similar and are adapted to generate a high-quality vapor medium that is emitted from the working end of the vapor-based device for ablating tissue.

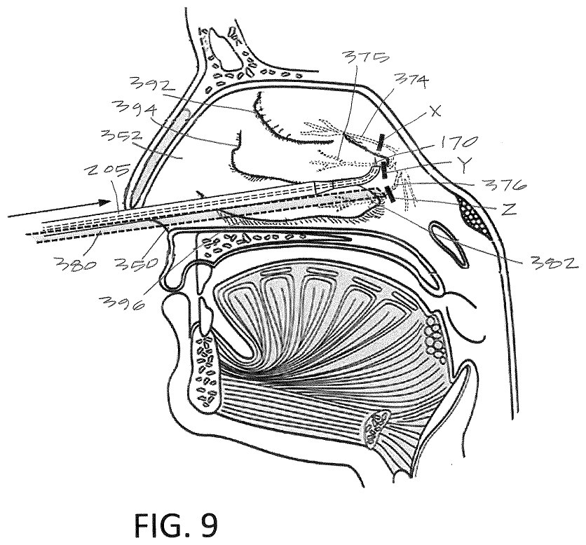

FIG. 1 depicts a handheld vapor-delivery device 100A configured for ENT procedures with its elongate introducer member 105 extending about axis 106 to a working end 112 configured for intraluminal positioning. The working end 112 can have several configurations and in the exemplary variation of FIG. 1, the introducer 105 and working end 112 are configured for introduction through a patient's nostrils and advanced to a position near the posterior nasal (PN) nerves to ablate the nerves to treat rhinitis (see FIG. 9). The device 100A is configured as a portable device suitable for use in operating rooms, ambulatory surgery centers (ASCs), or similar clinical environments.

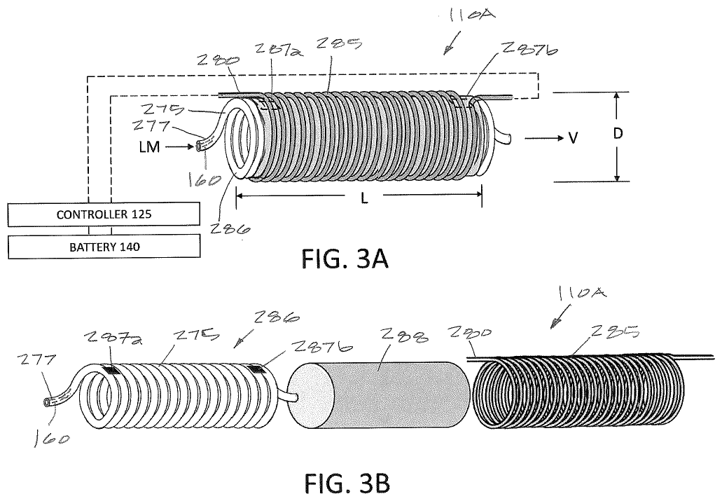

Referring to FIG. 1, the ergonomic pistol-grip handle 102 of the device 100A houses all functional subsystems of the vapor-generating unit are carried by the handle including a pre-filled volume of a liquid medium LM (such as sterile water) in syringe chamber 114, a pump subsystem 115 configured to provide fluid flow of the liquid medium LM, (which may comprise sterile water), and a resistive heating assembly or structure 110A configured to convert the flow of liquid medium LM into a flow of a vapor medium V. FIGS. 3A and 3B illustrate the resistive heating structure 110A of FIG. 1 in an enlarged view and an exploded view, respectively. Alternative types of resistive heating assemblies and architectures are described below.

In FIG. 1, the handle 102 further carries a controller 125 configured to control operating parameters of the pump subsystem 115 and the resistive heating structure 110A. The controller 125 can include, but is not limited to, a microcontroller, microprocessor, application-specific integrated circuit (ASIC), field-programmable gate array (FPGA), or any other suitable circuitry or programmable logic configured to execute a sequence of several tasks as described below.

The handle 102 also carries an onboard battery or battery pack 140 configured as the primary and exclusive electrical power source for the device 100A during a treatment procedure. The battery 140 is selected and integrated to enable fully self-contained operation of the handheld device 100A while delivering transient high-current electrical power sufficient to drive both the pump subsystem 115 and vapor-generating resistive heating structure 110A without reliance on external power supplies or wired conduits connected to remote consoles.

As shown in FIG. 1, the elongate introducer member 105 may have a non-limiting outer diameter ranging from approximately 4 mm to 6 mm and a length suitable for intraluminal positioning and accessing a targeted tissue, for example, posterior nasal nerves (see FIG. 9). In one variation, the introducer member 105 comprises a thin-wall stainless steel or high-strength polymer tube extending along a longitudinal axis 106, wherein the introducer member 105 may be fixed or detachable from the handle 102.

### Pump Subsystems and Liquid Medium Flow

This section describes exemplary pump mechanisms and liquid medium delivery arrangements that may be used to provide a flow of the liquid medium LM into contact with a vapor-generating resistive heating structure. The embodiments described herein are not limited to any particular pump type, actuator configuration, or mode of operation.

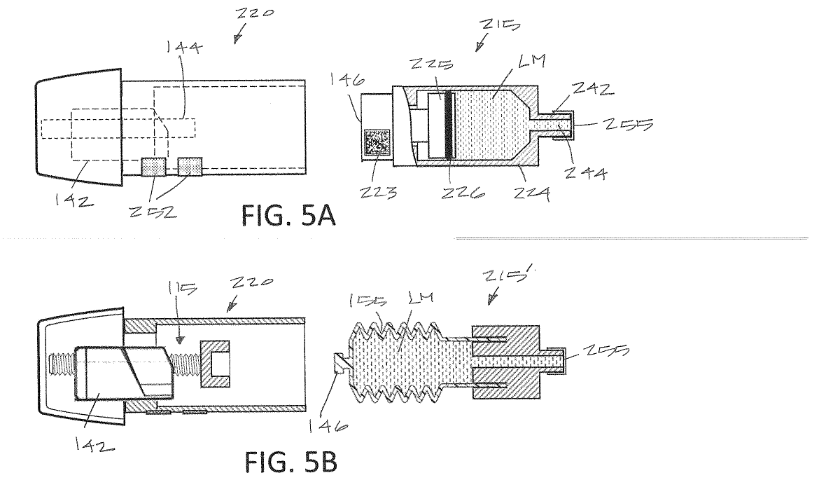

In a variation shown in FIG. 1, the pump subsystem 115 comprises a motor 142 driving a linear actuator 144 configured to engage a mechanical interface 146 of a movable plunger 148 in syringe 150 housing the syringe chamber 114 pre-filled with the liquid medium LM. The motor 142 may comprise a brushed DC motor, a brushless direct current (BLDC) motor, a stepper motor, or other electronically controlled motor suitable for battery-powered operation. The motor 142 is typically coupled to a planetary gear mechanism to provide gear reduction and increased output torque suitable for controlled displacement of the plunger 148 or controlled collapse of a deformable bellows 155 of the type shown in FIG. 5B.

In a method of operation, the motor-driven pump subsystem 115 is actuated by energizing the DC motor 142, which drives linear or compressive motion of the linear actuator 144 to engage the plunger 148 of the syringe 150 containing the liquid medium LM. Controlled actuation of the pump subsystem 115 delivers the liquid medium LM toward the heating structure 110A at the selected flow rate ranging from about 2 microliters/sec to about 20 microliters/sec. The liquid medium LM flows from syringe chamber 114 into the flexible flow channel 158 in fluid communication with vapor flow channel 160 extending through the resistive heating structure 110A to the working end 112. The liquid medium LM is heated and vaporized upon contact with the resistive heating structure 110A to generate the vapor medium V, which is conveyed through the flow channel 160 to at least one vapor outlet 165 (FIG. 2) and delivered into tissue. Release of latent heat of vaporization upon condensation of the vapor medium V, along with sensible heat, produces a therapeutic ablation effect in tissue.

### Flow Channel Configurations. Vapor-Delivery Needles, Surface Applicators

This section describes exemplary flow channel configurations and vapor-delivery working end configurations suitable for delivering the vapor medium V to interface with targeted tissue. The embodiments described herein are not intended to limit vapor outlet configurations, lumen dimensions, material selection, or needle geometries.

The vapor flow channel 160 extends through the resistive heating structure 110A and the introducer 105 to at least one vapor outlet 165 in the working end 112. In some variations, the flow channel 160 in the introducer is surrounded by thermal insulation or a concentric vacuum space. In a variation, the working end 112 includes at least one vapor delivery needle 170 (FIGS. 1, 2 and 4A) configured to be deployed into targeted tissue in an endoluminal position, for example through the wall of the nasal cavity into tissue carrying the targeted posterior nasal (PN) nerves (FIG. 9). In the variation of FIGS. 1 and 2, the vapor flow channel 160 extending through the needle 170 has an outlet 165 in the needle's sharp tip 172. In a variation, the needle 170 extends outwardly from the working end 112 at an angle ranging from 45° to 90° relative to the axis 106 of the introducer 105. The vapor-delivery needle 170 extending outward from the working end 112 may have a straight or curved configuration adapted to penetrate tissue and reach a targeted location. In a variation, the distal piercing portion of needle 170 has a size ranging from approximately 18 gauge to 36 gauge, suitable for penetration into nasal tissue. In certain variations, the needle shaft is formed from a high-temperature resistant polymer material, such as PEEK, Ultem, or other materials suitable for elevated-temperature vapor delivery, and may also comprise a metal needle. In the variation of FIG. 1, the axial core 174 may be rotated to align the direction of the needle 170 relative to the pistol grip of the handle 102.

Referring to FIG. 1, a trigger 175 is used to activate the device 100A to cause a flow of vapor medium V from at least one vapor outlet 165. Pressing the trigger 175 may deliver vapor for a preset time interval, for example, 1 second to 10 seconds, or may deliver vapor continuously while the trigger 175 is pressed. Other user-adjustable energy delivery options are described below.

FIGS. 4A and 4B illustrate the second embodiment of a vapor-based tissue treatment device 100B having a handle 202, introducer member 205, and working end 210. The device 100B is similar to the device 100A of FIG. 1, except that the liquid medium LM is carried in a chamber 212 of an insertable syringe cartridge 215 rather than in a fixed syringe chamber 114 within the handle 102 of FIG. 1. As can be seen in FIGS. 4A-4B, the resistive heating structure 110B is carried in a distal portion of the introducer 205. FIG. 4C shows the resistive heating structure 110B in an enlarged view, as further described below.

FIGS. 4A and 5A show the pre-filled liquid cartridge 215 that may be coupled to a drive housing 220, and the assembly is then inserted into the receiving channel 222 in the handle 202. An advantage of this cartridge-based variation is improved options for sterilization of the liquid media LM and the ability to replace the syringe cartridge 215 during a procedure if the volume of liquid medium LM is depleted. In another variation, the cartridge 215 may carry a liquid medium LM combined with a pharmacologically active agent that is delivered with thermal water vapor V as an adjunct therapy. In such cases, various insertable cartridges 215 containing different pharmaceutical agents and dosages may be provided for selection by the clinician. The syringe cartridge 215 is configured for single use and may optionally include an identifier 223 (FIG. 5A) readable by the device 100B to enable, restrict, or parameterize use of the liquid medium LM carried therein. The identifier 223 (FIG. 5A) also may be configured to be read by the controller 125 to verify cartridge compatibility, where the identifier 223 comprises a barcode, optical code, resistor, magnetic element, RFID element, or memory chip.

In the disassembled view of FIGS. 4A and 5A, the pre-filled syringe cartridge 215 comprises a body 224 defining the syringe chamber 212 that carries the liquid medium LM. A plunger 225 with O-ring 226 is configured to be advanced by the motor 142 and linear actuator 144 to move the liquid medium LM through a flow channel 240 into contact with the distal resistive heating structure 110B. As can be seen in FIGS. 4A and 5A, a distal tip 242 of the cartridge 215 includes a discharge outlet 244 sealed by a pierceable seal 245. The seal is pieced by needle 246 as shown in FIGS. 4A and 4B. The discharge outlet 244 is sealed prior to use and configured to open automatically upon insertion of the assembly of the cartridge 215 and drive housing 220 into the handle 202. The opening of the seal 245 irreversibly alters the cartridge to prevent re-use, and the syringe cartridge 215 is configured to be removed and discarded after a single treatment procedure. In other embodiments, the seal 245 comprises a frangible seal, a burstable seal, or a valve that is opened upon insertion of the cartridge 215 into the handle. The syringe cartridge 215 carries a predetermined volume of the liquid medium LM that ranges from about 2 cc to about 20 cc.

In FIGS. 4A and 4B, it can be seen that a drive housing 220 is configured to be insertable and removable relative to receiving channel 222 in handle 202. The drive housing 220 carries the DC motor 142 and linear actuator 144 components of the pump subsystem 115. The surface of the drive housing 220 includes electrical contacts 252 that couple to cooperating electrical contacts 254 in the receiving channel 222 of the handle 202, which are electrically coupled to the battery 140 to power the motor 142. The cartridge 215 and drive housing 220 may include alignment features configured to ensure proper orientation of the cartridge 215 in the drive housing 220. The drive housing 220 has alignment features, such as a key, to ensure proper orientation of the housing 220 with the receiving channel 222 to align the electrical contacts 252 and 254. A J-lock or similar locking mechanism may be provided to detachably lock the drive housing 220 in the handle. Referring to FIG. 4A, the cartridge 215 includes a mechanical interface 146 configured to engage the lead screw 144 of the pump mechanism, wherein the mechanical interface engages a lead-screw interface, a threaded engagement, a plunger shaft, a rack interface, a magnetic contact, or other push surface. FIG. 4B illustrates the liquid cartridge 215 and drive housing 220 assembled and inserted into the handle 202 of device 110B.

FIG. 5B illustrates another embodiment of a liquid media cartridge 215′ that is pre-filled with the liquid media LM contained in a collapsible bellows 155. The cartridge 215′ of FIG. 5B is similar to cartridge 215 of FIGS. 4A and 5A and includes a mechanical interface 146 configured to engage the pump mechanism, wherein the mechanical interface 146 may comprise a lead-screw interface, a threaded engagement, a plunger shaft, a rack interface, or other push surface. In all aspects, the pre-filled liquid media cartridge 215 of FIG. 5A and cartridge 215′ of FIG. 5B function in the same manner.

### Vapor Generation Subsystem Configurations

In the variations of FIGS. 1 and 4A-4B, the battery-operated vapor ablation devices 100A and 100B include vapor-generating resistive heating structures 110A, 110B, respectively, that are configured to convert a flow of a liquid medium (e.g., sterile water) in tubing 275 into a condensable vapor for delivery to a treatment site. FIG. 6 illustrates another variation of resistive heating structure 110C that comprises a primary heating structure 111A dimensioned for positioning in a device handle and a secondary heating structure 111B carried in a distal portion of an introducer.

In each of variations of resistive heating structures 110A-110C, the structure comprises a metallic tubing 275 with tubing wall 277 around the interior flow channel 160 (FIGS. 3B) and 240 (FIG. 4C) that defines at least a portion of the flow path of the liquid medium LM, wherein the tubing is thermally coupled to an electrically energized resistive wire 280 wound into a helical resistive heating element 285 (FIGS. 3B) and 290 (FIG. 4C). The tubing 275 may comprise a high-temperature, oxidation-resistant, and corrosion-resistant material, including stainless steel, nickel-based alloys, nickel-chromium alloys, nickel-chromium-molybdenum alloys, or other similar material that may be electrically resistive. In certain non-limiting embodiments, the tubing 275 may comprise an alloy corresponding to Inconel 625 or similar material. By way of example, the tubing 275 may have an outer diameter and inner diameter in a range of about 0.050″ and about 0.035″, respectively, although other dimensions may be used. As will be described below, the vapor generation subsystem may comprise one or more vapor heating assemblies configured to heat a liquid medium LM flowing through a flow path 160, 240, and to generate vapor for emission from one or more vapor outlets 165 at or near a working end of the device.

### Type A: Compact Proximal Tubing Coil Vapor Heating Assembly

In the variation of handheld device 100A shown in FIG. 1, a first non-limiting configuration (Type A) of resistive heating structure 110A is shown that includes tubing 275 with flow channel 160 therein in a compact form carried in the handle 102. As shown in FIGS. 3A and 3B, the tubing 275 may be formed into helical coil 286 configuration to provide a compact heated flow path with increased surface area and controlled residence time. The coil 286 may have an outer diameter D of less than about 15 mm, less than about 10 mm, less than about 6 mm, or as small as about 4 mm, and may have an axial coil length L of less than about 4 cm, or less than about 30 mm, or less than about 20 mm (FIG. 3A). The tubing coil 286 may be wound as a single-layer and may comprise a concentric multi-layer helix and may include any suitable pitch or turn spacing. At least one temperature sensor is coupled to the coil 286, with two sensors 287a and 287b shown in FIGS. 1 and 3B. The temperature sensors are adapted to send temperature signals to the controller 125 as described below. A further temperature sensor 287c may be positioned in the flow channel 160 distal to the heating structure 110A (FIG. 1).

In a variation shown in FIGS. 3A and 3B, electrical energy is delivered to the tubing coil 286 by the external resistive alloy wire 280 comprises a nickel-chromium-based alloy such as NiChrome or the alloy Kanthal formed into a helically wound resistive heating element 285 positioned around the tubing coil 286. A suitable resistance alloy for wire 280 has an electrical resistivity at room temperature ranging from approximately 1.0×10⁻⁶ ohm-meters to approximately 1.5×10⁻⁶ ohm-meters. The resistive alloy wire 280 may have a diameter ranging from 0.005″ to 0.025″ and wound at a tight pitch around the tubing coil 286. The resistive heating element 285 may optionally be separated from the metallic tubing coil 286 by a thin electrically insulating layer, such as a ceramic coating 288, wash, or sleeve to prevent electrical coupling while maintaining efficient thermal transfer (FIG. 3B). The resistive wire coil 285 is operatively coupled to the controller 125 and battery 140. The assembly 110A may further be surrounded by an insulation layer or thermal barrier within the handle 102 to reduce heat loss and improve battery-powered efficiency.

### Type B: Distal Straight-Tube Vapor Heating Assembly

In a second non-limiting configuration (Type B), the vapor heating assembly 110B of FIGS. 4C-4D is positioned in a distal region of the introducer member 205. In this variation, the heating assembly 110B may comprise straight tubing 275 with a resistive wire coil 290 or jacket positioned around a single straight tubing, or one or more parallel tubes defining a distal heated flow path therein. The resistive wire 280 of the coil 290 may be NiChrome or Kanthal as described above. The tubing 275 dimensions and material may be similar to those described above (e.g., about 0.050″ OD and about 0.035″ ID). The distal heating assembly 110B may include an insulation layer surrounding the resistive wire coil 290 to limit heat loss to adjacent structures and to enable compact integration near the working end 210. The resistive wire coil 290 is operatively coupled to the controller 125 and battery 140. Temperature sensors 287a and 287b are again shown coupled to the tubing 275 proximal and distal to the vaporization region that is inward of the coil 290.

Positioning a distal heating assembly 110B within the distal portion of the introducer member 205 as shown in FIG. 4A may reduce thermal losses along an intervening flow path and may reduce dead volume between a vaporization region and the vapor outlet 165, thereby improving vapor delivery response and repeatability.

### Type C: Primary Heating Assembly With Secondary Distal Vapor Conditioning Assembly

In a third non-limiting configuration (Type C) shown in FIG. 6, the device 100C carries a heating assembly 110C that includes both (i) a primary vapor heating assembly 111A positioned within a handle (e.g., the Type A compact tubing coil) and (ii) a secondary distal vapor heating assembly 111B positioned near the distal end of an introducer member 205 (e.g., the Type B straight-tube heater).

In such a variation, the primary heating assembly 111A may provide a majority of the thermal energy required to convert the liquid medium LM into vapor, while the secondary distal heating assembly 111B may operate as a vapor conditioning or finishing heater configured to deliver additional thermal energy to vapor V flowing toward the vapor outlet 165. The secondary distal heating assembly 111B may, for example, increase vapor quality, reduce liquid droplet carryover, suppress two-phase instabilities, offset heat loss along the introducer, or provide a controlled degree of superheat prior to emission from the vapor outlets 165.

The primary heating assembly 111B in Type C may optionally be operated at lower peak power than would be required in the absence of the secondary distal heating assembly 111B, thereby improving compatibility with an onboard battery power source.

In the Type A to Type C variations above, the flow channel 240 with the resistive heating structure 110C is within tubing 275 wherein the tubular heating wall 277 provides a substantially continuous wetted surface for heat transfer. The thermal mass of the tubing wall 277 acts as a heat reservoir that smooths temperature fluctuations during pulsed operation and promotes stable vapor generation with reduced liquid droplet carryover. In some variations, the inner surface of tubing wall 277 may be smooth to reduce flow disruption and undesired nucleation along the flow channel. In other variations, the inner surface of the wall 277 may include a microtextured or micropatterned surface region configured to provide nucleation sites that promote controlled vaporization and efficient phase change of the liquid medium LM within a selected vaporization zone. Thus, surface finish and microstructure of the surface of the flow channel may be selected to achieve a desired balance of vapor quality, nucleation behavior, and two-phase flow stability.

In the Type A to Type C variations above, the resistive heating coils 285 (FIGS. 3B) and 290 (FIG. 4C) are formed from a resistance alloy wire 280 or other electrically resistive metallic heating material selected to provide an electrical resistivity at room temperature in a range from about 1.0×10⁻⁶ Ω·m to about 1.5×10⁻⁶ Ω·m, including nickel-based resistance alloys, iron-based resistance alloys, stainless steels, or combinations thereof.

The resistive heating coils 285, 290 are electrically coupled to the onboard battery and configured to rapidly convert electrical energy into thermal energy for vaporization of the flow of liquid media LM in tubing 275. In certain implementations, the onboard battery 140 and resistive heating coil cooperate to deliver a transient electrical current of at least 3 Amperes to the resistive heating coil during vapor generation. The transient current delivery may occur during short vapor delivery intervals to support rapid heating and efficient phase conversion of the liquid medium. In some embodiments, the transient electrical current delivered to the resistive heating coil may be at least 5 amperes, at least 8 amperes, at least 10 amperes, or other suitable peak current levels, depending on the vapor generation rate and the battery discharge characteristics.

The controller 125 is configured to coordinate operation of the pump subsystem 115 with energization of the resistive heating coil 285, 290 such that flow of liquid medium LM and transient electrical current delivery occur in a synchronized manner to convert the liquid medium into a condensable vapor V. In further embodiments, the resistive heating coil 285 (FIGS. 3B) and 290 (FIG. 4C) may have an overall electrical resistance in a range from approximately 1 ohm to approximately 5 ohms, selected to permit high instantaneous heating power under battery operation while maintaining safe current draw limits. The vapor V is then delivered through the flow channel extending to a working end having one or more vapor outlets for application to target tissue.

### Illustrative Flow Rates and Power Levels

In certain embodiments, the vapor heating assembly or assemblies 110A-110C are configured to vaporize a liquid medium LM comprising sterile water at a flow rate in a range from about 2 μL/s to about 20 μL/s. By way of non-limiting example, heating liquid water from approximately 20° C. to approximately 100° C. and vaporizing the water at atmospheric pressure may correspond to a theoretical energy input of approximately 2.59 kJ per gram of water. Accordingly, vaporizing water at about 5 μL/s (~0.005 g/s) may correspond to a theoretical power of approximately 13 Watts, and vaporizing water at about 10 μL/s (~0.010 g/s) may correspond to a theoretical power of approximately 26 Watts. In further embodiments, electrical power applied to the vapor heating assembly may exceed theoretical vaporization power to account for heat losses, thermal mass, insulation effectiveness, duty cycle, system pressure, and control margin, such as by operating at about 15 Watts to about 60 Watts for flow rates within about 5 μL/s to about 20 μL/s, depending on the desired vapor quality or degree of superheat.

In certain embodiments, vapor is generated and delivered in a plurality of discrete pulses. During each pulse, electrical power from the battery 140 is applied to the resistive heating structure (110A-110C) to generate vapor, followed by a reduced-power or de-energized interval between pulses. The compact heating assemblies described herein may enable rapid initiation and termination of vapor generation with reduced overshoot and improved pulse-to-pulse repeatability while maintaining reliable phase change at flow rates in a range from about 2 μL/s to about 20 μL/s. The foregoing configurations, dimensions, materials, and operating parameters are provided as illustrative, non-limiting examples, and other heater geometries, locations, materials, and power levels may be used to achieve a desired vapor output for tissue treatment.

### Flow Stabilization Features for Vapor Quality and Repeatability

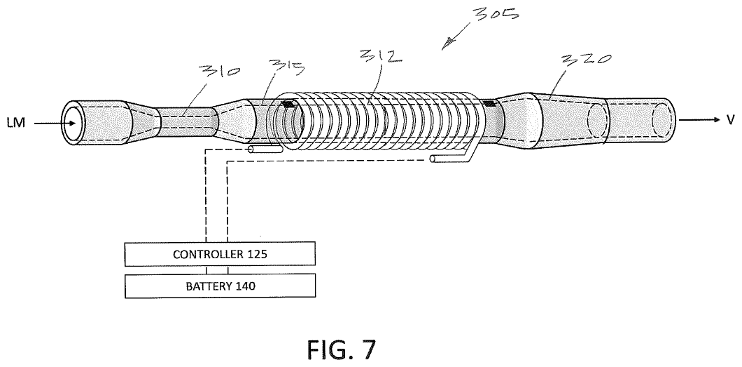

In certain embodiments, the vapor generation subsystem 110A-110C may further include one or more flow stabilization features associated with either or both of a primary Type A vapor heating assembly (FIGS. 3A-3B) and a Type B distal heating assembly (FIG. 4C) to improve vapor quality and delivery repeatability. For example, a Type B heating assembly 305 is shown in FIG. 7 with an upstream flow restrictor 310 that is positioned proximal to a vaporization region 312 to provide a controlled pressure drop and to reduce flow oscillations associated with two-phase vaporization. The term two-phase vaporization as used herein refers to a condition in which the flow of liquid medium LM and vapor V co-exist contemporaneously during the phase-change process in the vaporization region 312 of the heating assembly 305. A uniform flow of vapor V is not created instantly upon the liquid medium LM flowing into the heating assembly, and the flow initially consists of a dynamic mixture of vapor bubbles, vapor bubble formation and collapse, and residual liquid medium droplets that result in intermittent pressure fluctuations.

The proximal flow restrictor 310 can assist in generating high-quality vapor by stabilizing the upstream pressure and flow conditions within the vaporization region 312. By increasing backpressure and damping any rapid pressure/flow oscillations associated with bubble formation and collapse, the flow restrictor 310 promotes more complete vaporization within the vaporization region 312 and reduces liquid droplet entrainment and thus provides a more uniform single-phase vapor V provided by the heating assembly 305.

In some non-limiting embodiments, the restrictor 310 may comprise a reduced-diameter passage having an inner diameter about 50% to 80% the diameter of the flow channel 315 in the vaporization region 312. The length of the restrictor 310 may be from about 2 mm to about 20 mm, which may provide a pressure drop of approximately 2 psi to 8 psi at a liquid flow rate of about 6 μL/s as described previously. The restrictor 310 alternatively may comprise an orifice, a capillary segment, or a porous element. Additionally or alternatively, a downstream expansion zone 320 (FIG. 7) may be provided immediately distal to a vaporization region 312, such as a small cavity or enlarged volume having an inner diameter about 110% to about 200% the diameter of the flow channel 315, wherein the expansion zone 320 has an axial length of about 1 mm to about 5 mm. Such an expansion zone 320 may again dampen pressure fluctuations, reduce droplet carryover, and stabilize control any two-phase vaporization behavior prior to the flow of vapor V exiting the heating assembly 305 to propagate to the one or more vapor outlets 165 in the working end. In combination, the upstream restrictor 310 and/or downstream expansion zone 320 may assist in optimizing vapor generation, suppressing sputter or intermittent flow, improving the vapor fraction, and enabling more consistent pulsed vapor delivery in a battery-powered handheld system.

### Battery Power Source

In the embodiments of FIGS. 1 to 6, the devices carry vapor generation subsystems 110A, 110B and 110C that are designed around the electrical performance characteristics of an onboard power source comprising battery 140 or battery pack, such that the geometry, resistivity, placement, and duty cycle of the vapor-generating resistive heating structure, as well as liquid delivery parameters, are selected to operate within battery-defined voltage, current, and thermal limits while still achieving effective vaporization of a flow of liquid media LM.

In variations, the power source comprises a battery that may be rechargeable and ius configured to deliver sufficient electrical power to operate the pump subsystem 115, controller 125, and resistive heating structure 110A-110C during vapor generation. By way of example, the battery may be configured to provide peak power levels of approximately 10 Watts to 120 Watts at operating voltages ranging from approximately 3 Volts to 24 Volts, with transient current delivery on the order of 3 Amps to 20 Amps during vaporization cycles. Such power capacity enables multiple vapor delivery cycles per procedure, where vapor delivery intervals or pulses may range from 1 second to 10 seconds.

The battery 140 or battery pack is selected to provide high energy density, compact form factor, and reliable discharge characteristics suitable for the current demands of the resistive heating element. Suitable battery chemistries include, without limitation, lithium-ion (Li-ion), lithium-polymer (LiPo), and lithium iron phosphate (LiFePO₄) chemistries. In many embodiments, Li-ion batteries are preferred due to their high specific energy, typically in the range of 150 to 250 Wh/kg, and widespread availability in standardized formats.

Non-limiting examples of suitable Li-ion battery formats include cylindrical cells designated by dimensional codes corresponding to diameter and length in millimeters. In certain embodiments, the device incorporates one or more 18650 cells (approximately 18 mm×65 mm), which provide a balance of capacity (typically 2000-3500 mAh per cell) and compactness, allowing multiple cells to be arranged in series or parallel within the handle or within a removable battery pack. Higher-capacity alternatives include 21700 cells (approximately 21 mm×70 mm, typically 4000-5000 mAh) and 26650 cells (approximately 26 mm×65 mm, typically 4500-5500 mAh) for extended operating time or reduced recharge frequency. For more compact or lower-power embodiments, smaller cylindrical cells such as 16340 cells (approximately 16 mm×34 mm) may be employed, including in disposable or single-use handle configurations.

In general, the specific battery format is selected based on desired discharge capability, physical integration within the handle, and thermal management requirements, rather than on nominal capacity alone. These standardized battery formats offer high energy density, low self-discharge, and established safety features, including protection against overcharge, over-discharge, and short-circuit conditions, making them well-suited for powering resistive vapor-generation elements in nasal passageways or other body lumens.

The battery 140 or battery pack is housed directly within the device handle or may be provided in a removable battery pack to allow for charging and rapid insertion. In rechargeable embodiments, the device may include a charging interface, such as a USB-C port, magnetic connector, or other suitable coupling. In multi-cell configurations, a battery management system (BMS) may be provided to balance cell charging, monitor temperature and voltage, and ensure safe operation at the required discharge rates.

In certain variations, the controller 125 is configured to inhibit or terminate vapor generation based on one or more battery-related conditions, including insufficient state of charge, excessive discharge current, elevated battery temperature, or voltage outside a predetermined operating range. Such battery-aware interlocks enhance safety, ensure consistent vapor quality, and prevent incomplete vaporization associated with inadequate power delivery.

The controller 125 may be configured to coordinate operation of the pump assembly 115 and the resistive heating structure 110A-110C to deliver condensable vapor during one or more delivery intervals or pulses. In some embodiments, the controller 125 allocates an energy budget per pulse and controls both (i) a metered volume of liquid delivered by the pump assembly 115 and (ii) electrical power delivered to the resistive heating structure 110A-110C to satisfy the allocated energy budget while maintaining vaporization of the metered liquid volume.

The onboard battery 140 may define an operating voltage window, discharge current capability, thermal operating range, and state-of-charge characteristics. The device may further include one or more battery condition sensors configured to provide signals indicative of battery voltage, discharge current, battery temperature, state of charge, or combinations thereof. The controller 125 may be configured to control at least one operating parameter of the pump subsystem 115 and at least one operating parameter of electrical power delivered to the resistive heating structure 110A-110C based at least in part on a sensed battery condition.

In some embodiments, the controller 125 is configured to inhibit initiation of vapor delivery or to terminate an ongoing vapor delivery when a sensed battery condition satisfies a threshold condition associated with insufficient available power to maintain vaporization. Such inhibition or termination may occur before or during a delivery interval or pulse.

### Distal Positioning of Resistive Heating Structure

In certain embodiments, such as the vapor-delivery device 100B of FIGS. 4A-4B, the resistive heating structure 110B is positioned within the flow channel 240 at a location proximate the working end 210 and vapor outlet 165. Positioning the heating structure 110B close to the vapor outlet(s) 165 significantly reduces the internal flow path length between the vaporization region of the heating structure 110B and the tissue interface. For example, the distal end of the heating structure 110B of FIG. 4A may be less than 50 mm from the vapor outlet, or less than 40 mm, or less than 30 mm. By reducing the distance that vapor medium V must travel prior to emission from the vapor outlet 165, the volume of liquid and vapor within the flow channel 240 that does not immediately contribute to tissue ablation is minimized. This reduction decreases thermal losses associated with reheating residual liquid, re-vaporizing condensed fluid from prior delivery cycles, and conductive heat loss along the length of the flow path and a needle. As a result, a greater fraction of the thermal energy generated by the resistive heating structure is delivered to tissue in the form of latent heat of vaporization.

Because thermal energy is generated close to the needle and delivered over a short flow path, effective vapor generation and therapeutic ablation may be achieved using substantially lower electrical power levels than systems in which vapor is generated in the handle, as in FIG. 1.

In particular, distal placement of the heating structure 110B reduces the need to maintain elevated vapor temperatures over extended internal conduits and eliminates or reduces the need for complex thermal insulation in the introducer member. In some embodiments, the resistive heating structure 110B is configured to vaporize a controlled flow of sterile water using peak electrical power levels on the order of tens of Watts or less, while maintaining consistent vapor quality and delivery duration. The reduced power requirement enables compact power electronics, simplified thermal management, and integration of the vapor-generation subsystem of FIGS. 4A-4C within the handheld device.

Additionally, the short, heated length and reduced dead volume associated with distal placement of the heating structure 111B enables rapid onset and termination of vapor delivery, allowing precise control of vapor dose and energy delivery per unit time and further improving efficiency while reducing wasted energy. Accordingly, placement of the resistive heating structure 111B in the distal region of the introducer 205 provides a technical basis for effective vapor-based ablation at reduced electrical power levels while maintaining predictable and repeatable ablation performance.

### User-Adjustable Energy Delivery Control

In a variation, the handle includes a user-actuatable control, such as a button 325 (see FIGS. 1 and 4A-4B), switch, toggle, dial, or touch-sensitive interface, that allows a clinician to select between at least two different energy-delivery settings corresponding to different levels of thermal energy delivered to tissue per unit time. The selected setting may correspond to a desired caloric output/second or power level delivered by the device during vapor delivery.

Actuation of the user-adjustable control button 325 provides an input to the controller 125, which adjusts operation of the pump mechanism 115 and the current delivered to the resistive heating structure 110A-110C in a coordinated manner based on the selected setting. In particular, selection of a higher energy-delivery setting causes the controller 125 to increase a flow rate of the liquid medium LM delivered toward the heating structure 110A-110C while also increasing electrical current or power delivered to the heating structure to ensure complete vaporization of the liquid medium LM at the increased flow rate. Selection of a lower energy-delivery setting causes the controller 125 to reduce the flow rate and correspondingly reduce electrical current or power delivered to the heating structure 110A-110C.

In a variation, the controller 125 implements predetermined operating profiles or control tables that associate each selectable energy-delivery setting with corresponding liquid-medium flow rates and heating-structure power levels. In another variation, the controller 125 dynamically adjusts flow rate and heating-structure current using feedback from one or more temperature sensors 287a, 287b (FIGS. 3B, and 4C) to maintain vapor generation within a desired operating range for each selected setting.

The user-adjustable control may allow selection between discrete energy-delivery levels, such as a low setting and a high setting, or among three or more predefined settings. In all such variations, coordinated adjustment of liquid flow rate and heating-structure power ensures stable generation of the vapor medium V while delivering a selected level of thermal energy to tissue.

In one exemplary, non-limiting implementation, a handheld vapor-delivery device was developed to deliver from 25 cal/sec to 50 cal/sec. Electrical power is supplied by a rechargeable lithium polymer battery, such as a 14.8-volt battery rated at approximately 450 milliamp-hours with a discharge capability of up to approximately 30 amps. In an example, the battery comprises a Spektrum SPMX4504550 battery developed for high-current applications. Battery voltage supplied to the resistive heating structure 110A (FIG. 1) is modulated using a pulse-width modulation (PWM) circuit controlled by the MCU on a printed circuit board (PCB). During operation, the electrical current delivered to the resistive heating structure is approximately 10 amps, corresponding to approximately 120 watts of electrical power delivered to the heating structure having a resistance of approximately 1.2 ohms. Calorimetric measurements indicated an output of about 25 cal/sec, yielding an overall power conversion efficiency of approximately 88 percent. Battery discharge during a ten-second vapor delivery pulse at 10 amps corresponds to approximately 100 amp-seconds, or approximately 28 milliamp-hours, enabling energy delivery pulses when aggregated of about 150 to 180 seconds, which typically may consist of pulse intervals ranging from 1 to 10 seconds. In a variation, the battery-powered device is configured to deliver vapor medium from the working end for at least 60 seconds, at least 120 seconds, or at least 180 seconds.

In an exemplary configuration, the 25 cal/sec vapor delivery corresponds to a liquid medium LM flow rate of about 6 microliters/sec. Linear actuator speed and force requirements were selected to accommodate nominal operating pressures of approximately 15-20 psi and maximum pressures up to approximately 60 psi. The linear actuator used a current under maximum pressure conditions of less than approximately 0.1 Amp, which is negligible compared to the current delivered to the resistive heating structure 110A. The handheld device may include additional features such as LED indicators for power and operating state, fault indicators, a compact PCB with an embedded MCU, recharging interfaces including wired or inductive charging, and selectable vapor delivery pulse intervals.

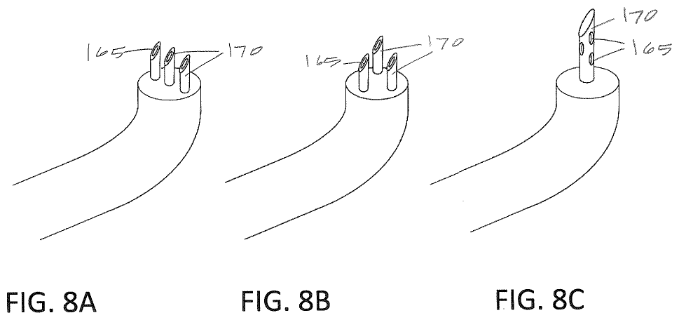

In other variations, FIGS. 8A-8C are illustrations of working ends configured with multiple needles 170 and a needle with a plurality of vapor outlets 165 along an outer surface of a needle tip. The number of vapor outlets, 165, may range from approximately 1 to 12.

Now turning to FIG. 9, a method corresponding to the invention is shown wherein the introducer 205 of the treatment device 100B of FIGS. 4A-4B is introduced through the patient's nostrils 350 into the nasal cavity 352 to the region of the posterior nasal (PN) nerves 374, 375, and 376. The clinician then punctures the needle 170 into the tissue overlying the targeted PN nerve. Next, the clinician actuates the system 100B by pressing trigger 175 (FIG. 4B) to deliver vapor medium V into tissue to thereby ablate the targeted PN nerve. In a variation shown in FIG. 9, the clinician utilizes an additional diagnostic tool with an elongated shaft w that is introduced into the nasal cavity 352 to contact the device working end 382 to the region of targeted tissue. The working end 382 carries a Doppler sensor mechanism that can locate blood vessels that can be avoided. Thereafter, the needle 170 can be penetrated toward the targeted PN nerve away from any blood vessel located within the Doppler sensor mechanism. The Doppler sensor mechanism can comprise either an ultrasound transducer, known in the art, or a laser light Doppler sensing mechanism, as is known in the art. In another variation, the working end 382 of the diagnostic tool 380 can also carry an image sensor for viewing the targeted treatment area. Such image sensors are well known in the art of endoscopes, with such image sensors having a diagonal dimension as small as about 1 mm. Further, the working end 382 can also carry an LED for illuminating the treatment area. In FIG. 9, the targeted treatment areas proximate to the PN nerves are shown as X, Y, and Z, where the targeted PN nasal nerves 374, 375, and 376 extend to the turbinates 392, 394, 396. In advance of vapor delivery, the clinician applies a topical anesthetic to the targeted tissue region. Vapor is then delivered for a very brief interval, typically from 1 second to 6 seconds, to ablate the targeted PN nerve, which results in reduction or elimination of nerve function, which in turn reduces or eliminates mucus hypersecretion. The method may further comprise the targeting of a plurality of PN nerves in the nasal cavity 352 or at least one PN nerve in a contralateral nasal cavity.

The method of the invention may comprise determining the location of the targeted PN nerve by one or more of the following targeting techniques, including (i) endoscopic determination of location based on the nasal anatomical landmarks; (ii) electrical neuro-stimulation of a targeted PN nerve while observing the physiological response to such electrical stimulation; (iii) providing an electrical nerve blockade while observing the physiological response to such a blockade, or (iv) identification of an artery associated with the targeted PN nerve using an ultrasonic or optical doppler flow technique.

In another variation, the vapor treatment device may have an introducer member 205 with a distal end carrying an expandable structure, such as a balloon, in a deflated configuration and an expanded configuration, wherein the balloon is dimensioned for placement against a lateral nasal wall proximate to a PN nerve, and can be expanded to press the working end into a targeted tissue surface. In another variation, the working end of a vapor treatment device may be carried on a first arm of a clamping assembly, wherein an opposing second arm of the clamping assembly and the first and second arms can be inside and outside of the nasal cavity or in the contralateral nasal cavity.

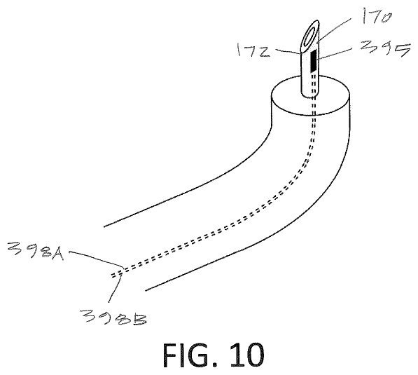

FIG. 10 illustrates another optional subsystem of the vapor delivery device comprising a bipolar capacitance mechanism or arrangement of bicap electrodes 395 near the distal tip of needle 170 as a component of a bicap system that functions to differentiate between different types of tissues based on electrical properties of tissue in contact with the bicap electrodes. As can be understood from FIG. 7, electrical leads 398A and 398B extend from the bicap electrodes 395 through the introducer 205 to the controller 125 (FIG. 4B). As background relating to the bicap system, various tissues in the body have different electrical properties due to variations in their composition and water content. For example, muscle tissue, fat, and blood vessels have distinct electrical conductivities and capacitances. In a variation, the pair of bicap electrodes 395 is in close proximity, with a first electrode functioning as a transmitter while the second electrode functions as a receiver. The transmitter electrode emits a low-energy electrical signal, typically in the form of an alternating current, into the engaged tissue, and the electrical signal interacts with the tissue's electrical properties, primarily capacitance. The response is influenced by the specific electrical properties of the tissue, such as its dielectric constant and capacitance. The receiver electrode coupled to the controller 125 measures the response of the tissue, and controller algorithms can then evaluate the changes in the electrical signal and its phase to differentiate between tissue types. The results can be displayed in real-time on a display (not shown), or an audible alert can be provided to indicate to the physician that the needle tip 172 is in contact with tissue targeted for ablation or, conversely, in non-targeted tissue.

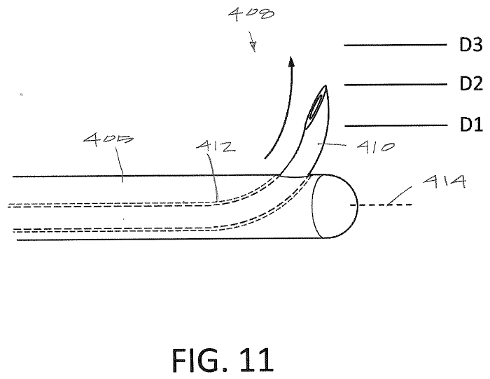

FIG. 11 illustrates another variation of an introducer 405 and working end 408 that carries an extendable and retractable needle 410. Such a needle 410 can comprise a flexible PEEK as described above with a sharp tip, wherein such a flexible needle can be actuated to move within a curved channel 412 in the introducer 405. Such an extendable needle 410 may be actuated by release of a spring-release mechanism in the device handle or motor drive mechanism to cause the needle to penetrate tissue. The needle 410 and needle drive mechanism may be configured to cause the needle 410 exit the introducer at 45° to 90°relative to the axis 414 of the introducer 405. In a variation, a spring-based needle drive mechanism includes a depth control mechanism to allow the needle 410 to penetrate tissue to a selected depth, for example, depths D1, D2, and D3 in FIG. 11.

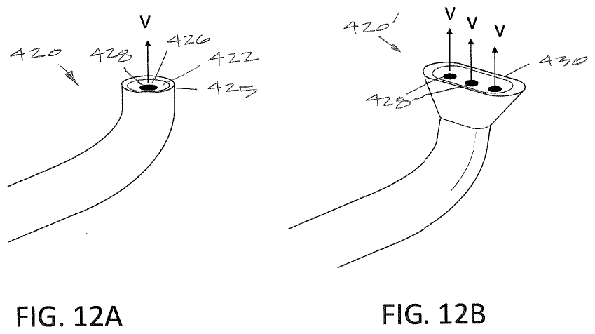

FIG. 12A shows another variation of a working end 420 with a surface ablation structure 422 that does not carry a needle. In this variation, the surface ablation structure 422 has a tissue-contacting perimeter 425 around a concavity 426 with a vapor outlet 428 therein. In a method of use, the clinician presses the tissue-contacting perimeter 425 against tissue overlying the targeted PN nerve and applies vapor for 1 to 5 seconds to ablate and desensitize the targeted PN nerve. The surface ablation structure 422 typically has a concave shape, and optionally, the concavity 426 may be configured with at least one vapor outlet 428. In another variation for other applications, the concavity may include an outflow port and outflow channel (not shown) for extracting condensate from the tissue surface. In a variation, the perimeter 425 may have suction ports for suctioning the perimeter against the tissue overlying the targeted PN nerve. FIG. 12B shows another variation of a working end 420′ with surface ablation structure 430 that is elongated and carries a plurality of vapor outlets 428. The concave structures 422, 430, and/or perimeters of FIGS. 12A and 12B may be rigid, semi-rigid, or compliant.

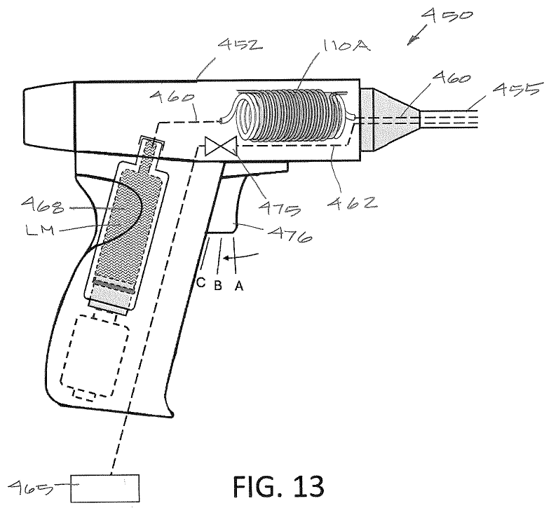

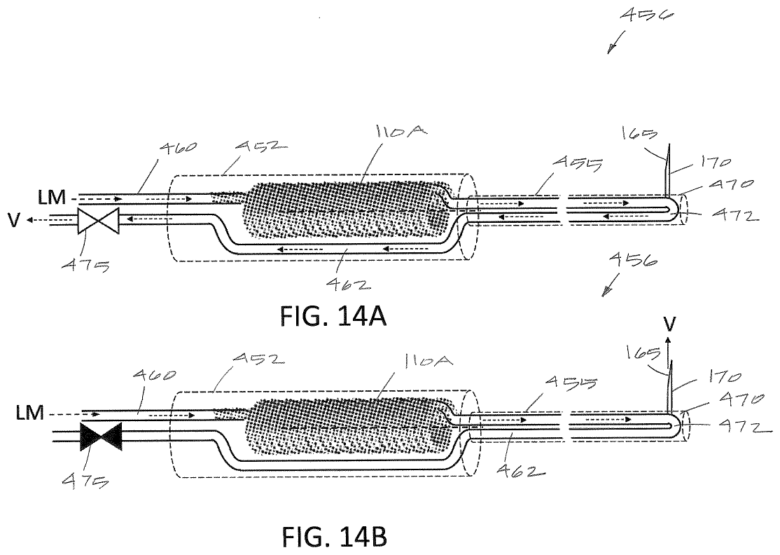

FIGS. 13, 14A and 14B illustrate another variation of a handheld vapor device 450 with handle 452 and introducer member 455 with working end 456 (FIG. 14A) that is similar to the device 100A of FIG. 1 with the addition of a vapor circulation system that allows for delivery of a vapor medium V substantially without any the delivery of any liquid condensate from a vapor outlet 165 in brief energy application intervals, for example, 1 second to 4 seconds as may be used in the ablation of PN nerves as depicted in FIG. 9. In some variations, such as the device of FIG. 1, there can be condensation in the flow channel 160 between the resistive heating structure 110A and the vapor outlet 165 in the needle 170. As can be seen in FIGS. 13 and 14A-14B, the device 450 is configured with an inflow channel 460 and an outflow channel 462 extending through the handle 452, introducer member 455, and working end 456. The outflow channel 462 optionally is assisted by a negative pressure source to aspirate condensing vapor media V through the outflow channel for collection in a collection reservoir 465.

In this variation, the flow of liquid media LM is delivered from the syringe chamber 468 through an inflow channel 460 to the helical heating element 110A, and wherein vapor medium V is delivered to the working end 456 (FIG. 14A) as described previously. In this variation, as can be seen in FIG. 14A, the vapor medium V flows from the heating structure 110A through the inflow channel 460 to the distal tip 470 of the introducer 455, then reverses flow in the reversing channel portion 472, wherein the flow of vapor medium V reverses direction from the inflow channel 460 into the outflow channel 462. Thus, FIG. 14A illustrates a circulating flow of vapor media V through the inflow channel 460, reversing flow channel portion 472, and outflow channel 462. In FIG. 14A, it can be understood that the lumen in the needle 170 is open to the inflow channel 460, but the cross-section of the needle lumen is much smaller than the cross-section of the inflow and outflow channels 460, 462 so that the vapor medium V will not flow through the lumen the of needle 170 but simply flow in the recirculating path through the outflow channel 462 which is the path of least resistance.

FIG. 13 shows a valve 475 in the outflow channel 462 actuated by trigger 476 in the handle 452. In FIG. 13, the valve 475 is in a normally open position to allow for circulating flow of fluid media through the device 450. In FIG. 14, the trigger 476 is in position A when the system is inactive, and the valve 475 is open. The clinician then can actuate the trigger 476 to position B, which activates the controller 125, pump subsystem 115, and heating structure 110A to generate the flow of vapor medium V with the valve 475 maintained in an open position, thus causing the vapor to flow in a circulating path through the inflow and outflow channels 460, 462. Thereafter, the clinician can actuate the trigger 476 from position B to position C, which closes the valve 475 and then results in vapor flowing through the lumen of the needle 170 and outward from the vapor outlet 165 to ablate targeted tissue. Thus, in the method shown in FIGS. 14A-14B, only high-quality vapor will exit the vapor outlet 165 in needle 170, which is important for treating or ablating PN nerves since only a brief interval of vapor delivery is needed.

For example, it is believed that only from 1 to 4 seconds of vapor delivery is needed to ablate PN nerves as depicted in FIG. 9. In the earlier variation of FIGS. 1 and 2, actuation of the system 100A could deliver condensate in the first second or a few seconds of operation as the flow channel 160 in the introducer 105 before the flow of vapor media V was emitted from the needle 170. The recirculating flow in the device 450 and FIGS. 13-14B solves this problem by circulating vapor through the inflow and outflow channels 460, 462 until vapor delivery is needed. In the variation of FIGS. 13-14B, the cross-sectional dimension of the lumen in the needle 170 is less than 50% of the cross-sectional dimension of the inflow and outflow channels 460, 462 to ensure that little or no vapor exits through the needle lumen when a circulating flow mode is activated. Often, the cross-sectional dimension of the needle lumen is less than 40% or less than 30% of the cross-sectional dimension of the inflow and outflow channels 460, 462.

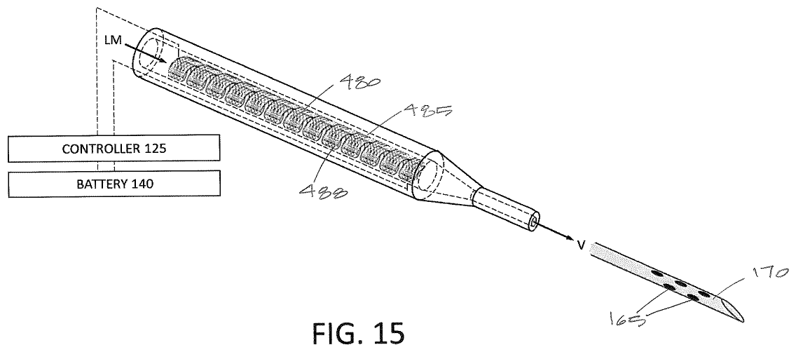

In another variation of the resistive heating structure that may be used, FIG. 15 illustrates a resistive heating element 480 positioned within a flow channel 485 in the handle or introducer. In such embodiments, the heating element 480 may comprise one or more electrically resistive wires or filaments 488, which may be formed into a helical geometry. The specific geometry of the resistive heating element may be selected to increase the effective heat-generating surface area within a confined volume of the flow channel 485 while remaining compatible with small-diameter channels configured to convey the liquid medium LM.

In the Type A, Type B, and Type C heating structure variations described above, the inner surface of the tubing 275 (see FIG. 6) provides a substantially continuous wetted heating surface during operation. The distributed nature of electrical resistance along the length of such tubing 275 results in relatively uniform heat generation and heat flux along the flow path. In addition, the tubular configuration may provide a degree of thermal inertia that can moderate temperature fluctuations in the tubing wall during changes in operating conditions, such as initiation or termination of vapor delivery.

In the embodiment of FIG. 15, the resistive heating element 480 comprises one or more elongated resistive wires 488 arranged within the flow channel 485. In some variations, multiple resistive filaments are arranged in a multifilar or helical configuration, and the liquid medium flows around and between the filaments as thermal energy is generated. In such wire-type heating configurations, vapor generation behavior during initiation of liquid flow and application of electrical power may differ from tubular heated-wall configurations. For example, localized bubble nucleation, intermittent liquid-vapor mixing, or transient two-phase flow effects may occur, which in some circumstances can contribute to sputtering or reduced vapor quality.

Accordingly, resistive heating structures, such as the Type A-C embodiments, that provide a substantially continuous heated wall surrounding the flow channel may promote more uniform vapor generation and may reduce liquid droplet carryover, thereby facilitating delivery of higher-quality condensable vapor.

In another variation, a handheld device has a working end that is deflectable relative to a longitudinal axis of the introducer, where the handle has a deflection mechanism as is known in the art to allow the user to selectively control the deflection. In this variation, the use can deflect the working end to position the needle or vapor outlet toward a targeted tissue region. In other variations, the working end can comprise clamps, forceps, and the like.

Although posterior nasal nerve ablation for treatment of chronic rhinitis as described above represents a principal application, the invention is not limited to that indication. The disclosed vapor-based devices and methods may be used to selectively treat or modify a variety of nasal, nasopharyngeal, or upper-airway tissues where controlled thermal delivery is desirable. Such tissues may include, without limitation, neural structures, submucosal tissue, glandular tissue, lymphoid tissue, or hypertrophic mucosa.

By way of non-limiting example, the technology described herein may be applied to conditions or procedures including inferior or middle turbinate hypertrophy, submucosal tissue reduction to improve nasal airflow, septal swell body reduction, nasal valve obstruction via adjacent tissue remodeling, vasomotor or non-allergic rhinitis, allergic rhinitis using nerve-targeting or submucosal approaches, adenoid or tonsillar tissue hypertrophy, nasopharyngeal lymphoid tissue reduction, chronic post-nasal drip associated with neurogenic or glandular targets, eustachian tube dysfunction involving treatment of tissue adjacent the tubal opening, soft palate or supraglottic tissue modification, and other selected upper-airway tissue treatments.

In all such embodiments, the disclosed systems enable precise control of vapor generation, delivery duration, and energy deposition to permit localized tissue treatment while minimizing unwanted thermal spread. The scope of the invention encompasses variations in device configuration, delivery interface geometry, treatment location, and target tissue type consistent with the principles described herein.

The vapor-generating architectures, needle structures, pump mechanisms, heating elements, control algorithms, energy-delivery schemes, and safety features described herein also may be combined in different ways depending on the intended clinical use, and no single embodiment or feature combination should be interpreted as limiting the scope of the disclosed inventions. Features described in connection with one embodiment may be used alone or in combination with features of other embodiments, including embodiments directed to different clinical indications. Accordingly, the present disclosure is intended to support claims directed to multiple related inventions, including but not limited to handheld ENT treatment devices and associated methods and systems.

While a number of exemplary embodiments have been described, it will be understood by those of ordinary skill in the art that various modifications, substitutions, and combinations may be made without departing from the spirit and scope of the present disclosure. Methods described herein may be performed in any order of steps that is logically possible, including orders different from those expressly described. In addition, steps or operations may be added, omitted, or combined to achieve desired results.

Unless otherwise indicated, the terms “comprising,” “including,” and “having” are used in an open-ended sense and do not exclude additional elements or steps. Numerical values, ranges, and dimensions described herein are approximate and may vary based on manufacturing tolerances, operating conditions, and design considerations. Features described in connection with one embodiment may be combined with features of other embodiments unless expressly stated otherwise.

Each of the embodiments and variations described herein may include features that are separable from, interchangeable with, or combinable with features of other embodiments. Modifications may be made to adapt a particular device, system, material, composition, or method to specific applications or objectives without departing from the scope of the present disclosure. As used herein, the term “about” refers to variations attributable to manufacturing tolerances, material composition, environmental conditions, and measurement uncertainty as understood by a person of ordinary skill in the art.

References to publications, patents, and patent applications are provided for informational purposes. Any such references are incorporated herein by reference only to the extent consistent with and not limiting the present disclosure.

The present disclosure is not limited to the particular embodiments described herein but is intended to encompass all modifications, equivalents, and alternatives falling within the scope of the appended claims. The scope of the disclosure further includes variations and embodiments that may become apparent to those skilled in the art in view of this disclosure.

1. A handheld vapor ablation device, comprising:
a handle carrying (i) a liquid medium source, (ii) a pump assembly, (iii) an onboard battery, and (iv) a controller;
a working end spaced from the handle and configured for positioning in contact with tissue;
a resistive heating structure thermally coupled to a flow channel fluidly coupled to the liquid medium source and configured to convert a liquid medium into a condensable vapor;
at least one vapor outlet at the working end and fluidly coupled to the resistive heating structure;
wherein the pump assembly is configured to deliver a controlled flow of liquid medium to the resistive heating structure at a flow rate ranging from about 2 microliters/second to about 20 microliters/second;
wherein the resistive heating structure has an overall electrical resistance of less than about 5 ohms;
wherein the controller is configured to coordinate operation of the pump assembly and energization of the resistive heating structure to deliver a condensable vapor in one or more delivery intervals; and
wherein the onboard battery comprises an exclusive electrical power source for the pump assembly, the resistive heating structure, and the controller during the one or more delivery intervals, such that the handheld vapor ablation device operates without a wired external power supply during vapor delivery.

2. The handheld vapor ablation device of claim 1, wherein the resistive heating structure comprises a resistive element in a helical form disposed within or about at least a portion of the flow channel.

3. The handheld vapor ablation device of claim 1, wherein the resistive heating structure comprises a resistance alloy having an electrical resistivity at room temperature ranging from approximately 1.0×10⁻⁶ ohm-meters to approximately 1.5×10⁻⁶ ohm-meters.

4. The handheld vapor ablation device of claim 3, wherein the resistance alloy comprises a nickel-chromium-based alloy.

5. The handheld vapor ablation device of claim 1, wherein each of the one or more delivery intervals comprises a vapor pulse having a duration ranging from about 1 second to about 10 seconds.

6. The handheld vapor ablation device of claim 1, further comprising a proximal flow restrictor positioned upstream of the resistive heating structure and configured to stabilize liquid flow and reduce two-phase flow instabilities during vapor generation.

7. The handheld vapor ablation device of claim 1, wherein the flow channel comprises a tubular member defining a substantially smooth inner wall configured to reduce flow disruption and promote stable vapor generation.

8. The handheld vapor ablation device of claim 1, wherein at least a portion of an inner surface of the flow channel comprises a microtextured or micropatterned region configured to provide nucleation sites to promote controlled boiling and vaporization within a selected vaporization zone of the flow channel.

9. The handheld vapor ablation device of claim 1, wherein the resistive heating structure comprises a resistive heating coil located within the handle.

10. The handheld vapor ablation device of claim 1, wherein the resistive heating structure comprises a resistive heating coil located in the introducer member at or near the working end.

11. The handheld vapor ablation device of claim 1, wherein the resistive heating structure comprises (i) a primary resistive heating coil located within the handle and (ii) a secondary resistive heating coil located in the introducer member, wherein the secondary resistive heating coil is configured to condition the condensable vapor prior to delivery from the at least one vapor outlet.

12. The handheld vapor ablation device of claim 11, wherein the secondary resistive heating coil is configured to reduce liquid droplet carryover.

13. The handheld vapor ablation device of claim 1, wherein the working end comprises at least one vapor-delivery needle.

14. The handheld vapor ablation device of claim 1, wherein the working end comprises a surface-contact applicator having a concave tissue-engaging geometry.

15. The handheld vapor ablation device of claim 7, wherein the tubular member comprises a metal tube, and wherein the resistive heating structure comprises a resistive coil disposed about an exterior of the metal tube such that heat is transferred through a wall of the metal tube to vaporize the liquid medium.

16. The handheld vapor ablation device of claim 15, wherein the resistive coil is not in direct contact with the liquid medium.
